(19) United States
(12) Patent Application Publication
(10) Pub. No.: US 2002/0122536 A1
(43) Pub. Date: Sep. 5, 2002

(54) Method and apparatus for viewing instrument markers used in medical imaging

(76) Inventors: Erwan Kerrien, Versailles (FR); Sylvie Jacquot Ingles, Puteaux (FR); Laurent Launay, Saint Remy les Chereuse (FR); Eric Maurincomme, Chevreuse (FR)

Correspondence Address: General Electric Company, 3135 Easton Turnpike - W3C, Fairfield, CT, 06431, US

(21) Appl. No.: 10/067,549

(22) Filed: Feb. 5, 2002

(30) Foreign Application Priority Data
Feb. 12, 2001 (FR) 0101859

(51) Int. Cl. H05G 1/28; A61B 6/08

(52) U.S. Cl. 378/205; 378/163

(57) ABSTRACT

Method and apparatus for viewing instrument markers used in medical imaging for calibrating a surgical or medical procedure assistance system. The apparatus comprises acquiring radiographic images, constructing a three-dimensional model from a set of radiographic images acquired, and locating, in a given coordinate system, at least one marker placed on an instrument used during the procedure. A set of opaque elements appearing on the images is placed in the field of the apparatus, and in that the position of a set of markers rigidly connected to the set of opaque elements is determined with respect to the locating coordinate system. A transformation is applied to the coordinate system for locating a marker whose position is automatically determined, depending on the positions thus determined for these markers in the coordinate system. The positions of the opaque elements on the images acquired or in the three-dimensional model reconstructed from these images, in order to show this marker in the coordinate system of the three-dimensional model.

### CROSS REFERENCE TO RELATED APPLICATIONS

This application claims the benefit of a priority under 35 USC 119 to French Patent Application No. 01 01 859 filed Feb. 12, 2001, the entire contents of which are incorporated by reference.

### BACKGROUND OF THE INVENTION

The present invention relates to a medical imaging apparatus and method and particularly to viewing the position of an instrument which is used on a three-dimensional model of the region of the patient.

Many procedures are already known for enabling three-dimensional models to be reconstructed from fluoroscopic images obtained by means of a channel gantry, or else by means of a device with C-shaped support arm. Radiologists and surgeons use this procedure to analyse pathologies and to prepare for a medical or surgical procedure. During a medical or surgical procedure, instruments are handled (for example biopsy needles, syringes, catheters, screws, etc.) using fluoroscopy for monitoring purposes. Means for locating display an image of the patient's body, on which image the position of the instrument used appears. The instrument is furnished with one or more markers to locate the instrument in space and the imaging system displays the position of this marker or these markers in the three-dimensional representation. These markers are, for example, radiofrequency sources, ultrasound sources, etc., the means for locating comprising a set of sensors capable of detecting the signals emitted by the markers and also comprising means for processing capable of calculating the distance between the sensors and the markers from the received signals and of carrying out a triangulation in order to calculate their position.

Alternatively, as shown in U.S. Pat. No. 5,967,980, the marker may be a receiver, the reference means comprising means for emitting, for example, an electromagnetic field.

Displaying the position of a marker in the three-dimensional representation provided by the imaging system assumes that the means for locating means are accurately calibrated with respect to the coordinate system of the three-dimensional representation. This calibration may, for example, be carried out manually by pointing out one or more particular points on the body of the patient (for example a projecting vertebra) using the instrument equipped with the marker and pointing out the corresponding point or points on the tomographic image.

However, it is easily understood that a manual calibration, apart from being tedious, is difficult to implement with a high degree of accuracy.

### BRIEF DESCRIPTION OF THE INVENTION

An embodiment of the invention is a method and apparatus which allows a particularly reliable and easy calibration of the coordinate system of a locating system with respect to the coordinate system of the reconstructed three-dimensional representation.

An embodiment of the invention is a method and apparatus for viewing instrument markers used in medical imaging for calibrating a surgical or medical procedure assistance system the apparatus comprises means for acquiring radiographic images, means for constructing a three-dimensional model from a set of radiographic images acquired by this device and means for locating, in a given coordinate system, at least one marker placed on an instrument used during the procedure, comprising a set of opaque elements appearing on the images are placed in the field of the apparatus, a position of a set of markers rigidly connected to the set of opaque elements is determined with respect to the locating coordinate system, a transformation is applied to the coordinate system for locating a marker whose position is automatically determined by the means for locating, depending on the positions thus determined for these markers in the locating coordinate system, and on the positions of the opaque elements on the images acquired or in the three-dimensional model reconstructed from these images, in order to show this marker in the coordinate system of the three-dimensional model.

In one embodiment of the invention, the opaque elements are elements placed in the field of the acquisition device during a calibrating step of the procedure for constructing the three-dimensional model, these opaque elements being rigidly connected to a set of markers. The opaque elements are preferably the opaque elements of a phantom such as a cylinder, used during a calibrating step of the procedure for constructing the three-dimensional model. The opaque elements are disposed on the phantom to form a helix. The cylinder carrying these opaque elements also carries the set of markers.

In another embodiment of the invention, the markers of the set used to calibrate the assistance system are opaque elements, these opaque markers being placed in the field of the acquisition device during a step of acquiring an image on a patient. In particular, the opaque markers can be placed directly on the patient.

An embodiment of invention also relates to a surgical procedure assistance system for comprising an apparatus comprising means for acquiring radiographic images, means for constructing a three-dimensional model from a set of radiographic images, means for locating, in a given coordinate system, at least one marker placed on an instrument used during the procedure, and means for processing by automatically implementing an embodiment of the disclosed method.

### BRIEF DESCRIPTION OF THE DRAWINGS

Other characteristics and advantages of the invention will emerge further from the following description, which is purely illustrative and non-limiting and can be read in conjunction with the appended drawings, among which:

FIG. 1 is a diagram illustrating one embodiment of the disclosed invention; and

FIG. 2 is a diagram illustrating another embodiment of the disclosed invention.

### DETAILED DESCRIPTION OF THE INVENTION

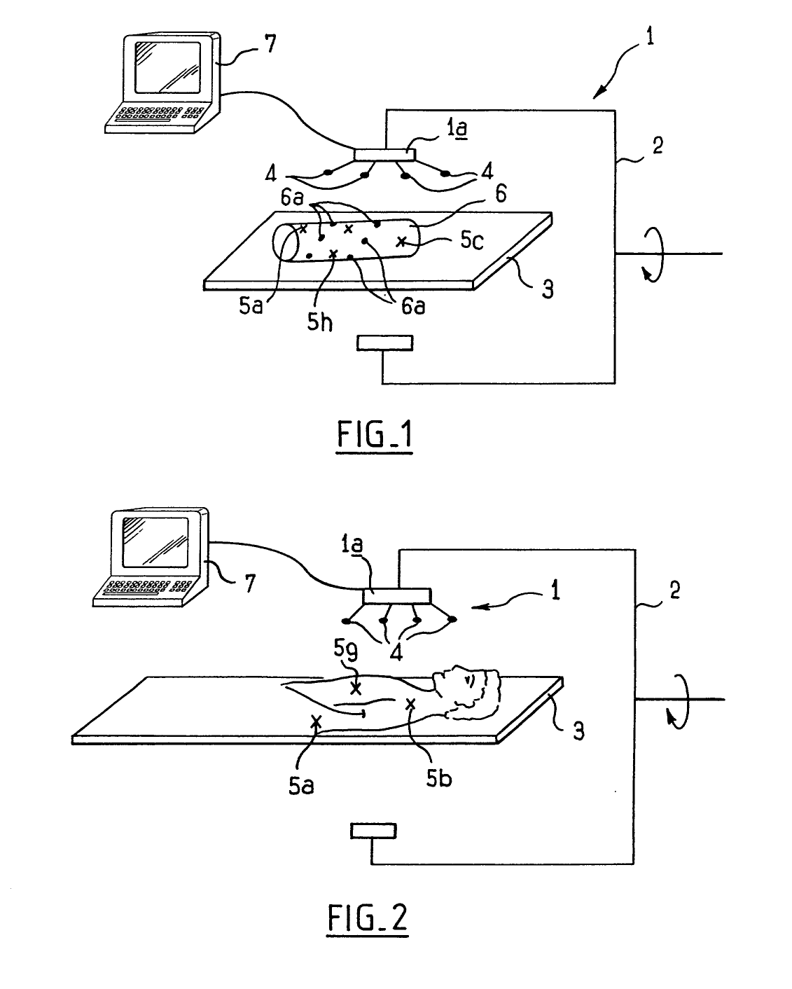

FIG. 1 shows an imaging device which comprises means 1 for acquiring images, for example, by X-ray fluoroscopy, mounted on a gantry 2 of a C-shaped-arm type capable of being rotated about an operating table 3 on which on abject, for example, a patient is disposed. Means for locating is provided so that one or more markers can be located with respect to a given coordinate system. In particular, a marker may be a radiofrequency source or receiver or an ultrasound source or receiver. The means for locating may comprise sensors or emitters for detecting signals emitted by the marker or of emitting signals intended therefore. The means for locating may comprising means for processing for determing the position of the marker according to the signals received, for example, by triangulation.

The coordinate system of the means for locating is, for example, a coordinate system fixed with respect to an operating theatre or with respect to the table 3. The means for locating comprises a coordinate system fixed with respect to the gantry 2, as illustrated in FIG. 1, in which a plurality of sensors 4 is attached to an X-ray emission module la. The position of the marker with respect to the coordinate system of the means for locating is therefore determined when the gantry 2 is stationary, for example, in its position as shown in FIG. 1.

According to one embodiment of the invention, a set of markers 5a, 5b and 5c (preferably at least three) are placed on a phantom 6 of the type of those which are conventionally used to calibrate a three-dimensional reconstruction with respect to the 2D images obtained by means for acquiring images. The phantom 6 comprises a cylindrical support made of plexiglas integrating a plurality of opaque point-like elements 6a distributed in a helical manner over the support. The calibrating procedure of the corresponding three-dimensional reconstruction is described in U.S. Pat. No. 5,442,674.

Once the phantom 6 is installed on the table 3, for example in the axis of rotation of the gantry 2 with a C-shaped arm, a set of images of the phantom 6 and of these opaque point-like elements is acquired. Means for processing 7 combined with the means for image acquisition implements a method described in U.S. Pat. No. 5,442,674. The implementation of this method by the unit 7 calculates a mathematical transformation (rotation/translation) to pass from the coordinate system of the three-dimensional reconstruction to a given coordinate system connected to the phantom 6. Moreover, the means for locating and the sensors 4 acquire, at the same time, the position of the markers 5a, 5b, 5c which are included with the phantom 6. Initially, knowing the position of these markers on the phantom 6, the means for processing 7 determines the transformation (rotation/translation) to pass from the coordinate system connected to the phantom 6 to the coordinate system of the locating device 4. By combining the two transformations obtained in this way, the means for processing 7 calculates the rigid transformation to represent a point whose coordinates are known in the coordinate system of the means for location, system, in the coordinate system of the three-dimensional reconstruction.

According to another embodiment of the invention, as illustrated in FIG. 2, the means for processing 7 determines this rigid transformation not during the calibrating procedure of the three-dimensional reconstruction with respect to the 2D images acquired, but during the procedure for acquiring images of the patient. The markers, 5a, 5b and 5c, are opaque elements and consequently, their position with respect to the coordinate system of the means for locating and whose position with respect to the coordinate system used for the three-dimensional reconstruction, will both be known. The markers are, for example, markers which are positioned on the object, for example directly on the skin of the patient, around the region of the surgical procedure, as illustrated in FIG. 2. The markers 5a, 5b, 5c may, for example, be adhesively applied directly on the skin of the patient. Since the image acquisition apparatus 1 has been calibrated in advance, the distortion field and the calibration matrix expressed in its coordinate system are known for each 2D image acquired by the apparatus.

A tomographic acquisition of the patient is then carried out. During this acquisition, N views are selected. The markers 5a, 5b, 5c are detected on each of these N images (images which are not subtracted, masked nor injected) and the two-dimensional coordinates of the markers 5a, 5b, 5c on the images corresponding to these views are recorded for these N acquisitions, then processed in order to calculate therefrom the three-dimensional coordinates of the markers in the coordinate system of the three-dimensional reconstruction. Moreover, the means for locating 4 for locating the markers supplies the coordinates of these markers 5a, 5b, 5c, in the coordinate system. Should the means for locating 4 be fixed in the operating theatre or connected to the table 3, it would still be in the reference position and the coordinates of the markers 5a, 5b, 5c would still be detected in the reference coordinate system of the locating apparatus.

After calculating, on the one hand, positions of these markers in the coordinate system of the means for locating, on the other hand, positions of these markers on the images corresponding to the N views (or else, which is equivalent, positions of these markers in the coordinate system used for the three-dimensional reconstruction), the means for processing 7 calculates the rigid transformation to pass from one position in the locating coordinate system to a position in the coordinate system of the three-dimensional reconstruction.

Various modifications in structure and/or steps an/or function may be made by one skilled in the art without departing form the scope and extent of the invention as recited in the claims.

What is claimed is:

1. A method for viewing instrument markers in an apparatus comprising:
means for acquiring radiographic images, means for constructing a three-dimensional model from a set of radiographic images; and
means for locating in a given coordinate system, at least one marker placed on an instrument used during a medical or surgical procedure comprising:
(a) placing a set of opaque elements appearing on the acquired images in the field of the provided by the means for acquiring images;
(b) determining a position of a set of markers connected to the set of opaque elements with respect to the coordinate system;
(c) applying a transformation to the coordinates in the coordinate system for locating a marker whose position is automatically determined by the means for locating, depending on the positions thus determined for the markers in the coordinate system, and on the positions of the opaque elements on the images or in a three-dimensional model reconstructed from the images, to show the marker in the coordinate system of the three-dimensional model.

2. The method according to claim 1 wherein the markers are rigidly connected to the opaque elements.

3. The method according to claim 1, wherein the opaque elements are placed in the field of the acquisition device during a calibrating step of the procedure for constructing the three-dimensional model, the opaque elements being connected to a set of markers.

4. The method according to claim 3 wherein the opaque elements are rigidly connected to the markers.

5. The method according to claim 3, wherein the opaque elements are opaque elements disposed on a phantom used during a calibrating step of the procedure for constructing the three-dimensional model, the phantom carrying the opaque elements also carrying the set of markers.

6. The method according to claim 5 wherein the opaque elements are disposed helically on the phantom.

7. The method according to claim 6 wherein the phantom is a cylinder.

8. The method according to claim 1, wherein the markers of the set are opaque elements, the opaque markers being placed in the field of the acquisition device during a step of acquiring an image on an object.

9. The method according to claim 8, wherein the opaque markers are placed directly on the object.

10. The method according to claim 9 wherein the opaque markers are adhesively applied to the object.

11. An apparatus for viewing instrument markers comprising;
means for acquiring a radiographic image;
means for constructing a three-dimensional model from a set of radiographic images acquired;
means for locating in a given coordinate system, at least one marker placed on an instrument used during a medical or surgical procedure;
means for placing at least one opaque element in the field of the acquired image; and
means for processing by transformation to coordinate in the coordinate system depending on the positions of the marker in the coordinate system, and on the positions of the opaque elements on the images or in a three-dimensional model reconstructed from the images, to show the marker in the coordinate system of the three-dimensional model.

12. The apparatus according to claim 11 wherein the marker is rigidly connected to the opaque element or vice versa.

13. The apparatus according to claim 11 wherein the opaque elements are opaque elements disposed on a phantom used during a calibrating step of the procedure for constructing the three-dimensional model, the phantom carrying the opaque elements also carrying the set of markers.

14. The apparatus according to claim 13 wherein the opaque elements are disposed helically on the phantom.

15. The apparatus according to claim 14 wherein the phantom is a cylinder.

16. The apparatus according to claim 11 wherein the markers of the set are opaque elements, the opaque markers being placed in the field of the acquisition device during a step of acquiring an image on an object.

17. The apparatus according to claim 16 wherein the opaque markers are placed directly on the object.

18. The apparatus according to claim 17 wherein the opaque markers are adhesively applied to the object.
